(19) United States
(12) Patent Application Publication
(10) Pub. No.: US 2025/0201416 A1
(43) Pub. Date: Jun. 19, 2025

(54) DEVELOPMENT AND VALIDATION OF THE HEMORRHOID DISEASE SYMPTOM IMPACT MEASURE

(71) Applicant: Cristcot LLC, Concord, MA (US)

(72) Inventors: Jennifer J. Davagian, Acton, MA (US); Mark C. Ensign, Newburyport, MA (US); Karthik Devarajan, Pearland, TX (US); Mukul Agrawal, St. Augustine, FL (US); Carolyn Schwartz, Concord, MA (US)

(73) Assignee: Cristcot LLC, Concord, MA (US)

(21) Appl. No.: 19/069,941

(22) Filed: Mar. 4, 2025

Related U.S. Application Data
(63) Continuation of application No. PCT/US2024/034869, filed on Jun. 20, 2024.
(60) Provisional application No. 63/509,415, filed on Jun. 21, 2023.

(51) Int. Cl. G16H 50/20; A61B 8/00; A61B 8/04; A61B 8/06; A61B 8/08; G16H 10/20; G16H 15/00; G16H 20/00; G16H 50/30

(52) U.S. Cl. CPC G16H 50/20; A61B 8/04; A61B 8/06; A61B 8/0891; A61B 8/488; A61B 8/5223; A61B 8/5292; G16H 10/20; G16H 15/00; G16H 20/00; G16H 50/30

(57) ABSTRACT

Disclosed is a method of assessing health of a patient that includes presenting a patient reported outcome measure (PROM) for at least partial completion by the patient about an illness, injury, or condition. The PROM including questions extending beyond a question about a symptom or sign of the illness, injury, or condition. The method further includes performing a calculation as a function of responses to the questions to assess the health of the patient. A particular embodiment includes correlating a result of the calculation with an objective measurement of the symptom or sign to produce patient reported outcome information for assessing the health of the patient.

### CROSS-REFERENCE TO RELATED PATENT APPLICATIONS

This application is a continuation of PCT/US2024/034869 filed Jun. 20, 2024, which claims the benefit of and priority to U.S. Provisional Application No. 63/509,415 filed Jun. 21, 2023, the entire disclosures of which are incorporated by reference herein.

### BACKGROUND

Hemorrhoid disease (HD) affects 10 million people in the U.S. at any given time, and 50% of the U.S. population (i.e., 167 million people) will develop symptomatic hemorrhoids during their lifetime. Approximately 60% of people with hemorrhoids experience symptoms. Despite its prevalence and impact on quality of life, the existing patient-reported outcome measures of HD have not been validated using standard psychometric methods.

### SUMMARY

An example embodiment of the present disclosure is a patient-reported outcome assessment system for use in HD clinical research, the Hemorrhoid Disease Symptom and Impact Measure (HD-SIM™ Assessment System). It began with a qualitative phase comprised of cognitive interviews with 16 patients. Over the course of four rounds of interviews, the item pool was generated and honed to elicit the best instructions, item wordings, and response options. The quantitative phase was a web-based survey of 1066 HD patients, with a subsample of 100 who also completed a retest survey 1-2 weeks later. The sample was stratified to be evenly distributed across mild, moderate, and severe levels of disease. The HD-SIM system includes 38 items representing six subscales: Symptoms at Worst, Symptoms at Best, Bowel Health Problems, Life Impact, Mental Health Impact, and Manageability. Psychometric analyses documented that the subscales had excellent internal consistency reliability, cross-sectional construct validity (i.e., convergent and divergent validity, known-groups validity), test-retest stability, and longitudinal construct validity (i.e., responsiveness). Combined, this psychometric evidence indicates that the HD-SIM system is a clinical research measurement tool that is fit for purpose for hemorrhoid disease symptom impact in clinical trial research.

A particular embodiment may be a method of assessing health of a patient by (a) presenting a patient reported outcome measure (PROM) for at least partial completion by the patient about an illness, injury, or condition, the PROM including questions extending beyond a question about a symptom or sign of the illness, injury, or condition; and (b) performing a calculation as a function of responses to the questions to assess the health of the patient.

The method may further comprise correlating a result of the calculation with an objective measurement of the symptom or sign to produce patient reported outcome information for assessing the health of the patient.

The questions may include questions about physical symptoms and at least one question selected from a group consisting of: questions specifically directed to bodily tissues impacted by the disease that is causing the physical symptoms, questions about manageability of the disease, questions about mental health impact caused by the disease or the physical symptoms, and questions about life impact resulting from the disease.

An example embodiment is directed to a hemorrhoidal disease, and the questions may include at least a subset of the following related to corresponding questions: (a) symptoms: pain during bowel movement (BM), pain not during BM, burning while urinating, itching, bleeding, anal leakage, discomfort, pain while sitting; (b) bowel health: plugged up, emptied bowels, number of times went to the bathroom, time in bathroom, clean anal area, and avoid BM while not at home; (c) manageability: able to cope, manageable symptom effect, treatment effect effective; (d) mental health impact: worried, aggravated, distracted, unpredictable, isolated, embarrassment, mood, self-esteem; (e) life impact: affect sleep, interfere with daily activities, interfere with enjoyable time with others, affect ability to take care of others, affect physical intimacy, and protect underwear. An example embodiment of views presenting such questions in HD-SIM is shown in FIGS. 3A-J.

An example embodiment may be directed to a hemorrhoidal disease, and the questions may include a question about whether burning while urinating is experienced or whether pain occurs during and/or not during bowel movements.

Example embodiments may include questions about the symptom or sign that prompt responses relating to the symptom or sign at its best and at its worst.

In an embodiment, the method performs a calculation that produces mean and standard deviation of unweighted scores based on the responses from the patient. The method may characterize severity of the illness, injury, or condition into categories of at least severe, moderate, and mild. Alternatively, the method may calculate a score along a continuum within an available range of the responses.

The method may be performed at a beginning and end of a study period, in which case the method may include taking at least one physical measurement of afflicted bodily tissues as at least part of the objective measurement at the beginning and the end of the study period. Correlating the result of the calculation may include correlating the patient reported output information at the beginning and end of the study period with the at least one physical measurement of the afflicted bodily tissues.

A treatment of the illness, injury, or condition may be applied during the study period, and the method may include determining if the afflicted bodily tissues correlate to progression of the illness, injury, or condition based on multiple physical measurements of the afflicted bodily tissues.

An example embodiment of the method may be applied to an illness that is hemorrhoidal disease and wherein the at least one physical measurement includes a measurement of blood flow through the hemorrhoidal vein or artery. The at least one measurement may include a Doppler measurement to quantify size, shape, and volume at the beginning and end of a study period.

The method may include reporting patient reported outcome information in a manner enabling selection of a treatment profile.

The method may be performed on multiple test patients that have been administered a drug or treated with a device and performed on multiple patients of a control population that have not been administered the drug or treated with the device, and wherein the method may further comprise reporting patient reported outcome information for both the multiple test patients and the multiple patients of the control population to a regulatory body responsible for approving the drug or device.

Another example embodiment is a method for assessing a state of hemorrhoidal disease. The example embodiment comprises: (a) observing varicosity in a region of tissues afflicted by the hemorrhoidal disease in a patient with a hemorrhoidal vein and artery; (b) measuring pressure and blood flow in the hemorrhoidal vein or artery over a length, width, and volume of the vein or artery; and (c) assessing a state of the hemorrhoidal disease as a function of at least one of the varicosity, pressure, and blood flow. The example embodiment may be performed at a beginning and an end of a treatment protocol period.

The method according to this example embodiment may further comprise: (a) obtaining, from the patient, responses to a patient reported output measure directed to the hemorrhoidal disease; (b) categorizing severity of the hemorrhoidal disease as a function of the patient responses to determine a category of the severity; and (c) comparing the state of the hemorrhoidal disease to the category of the severity. The method may further comprise recommending a treatment plan as a function of the results of the comparing.

Another example embodiment is a computer-implemented method for assessing health of a patient. This example embodiment includes: (a) enabling a patient to provide at least partial responses to a patient reported outcome measure (PROM) about an illness, injury, or condition via an electronic medium, the PROM including questions extending beyond a question about a symptom or sign of the illness, injury, or condition; (b) performing a calculation at the electronic medium, or at a server coupled to the electronic medium, as a function of responses to the questions; and (c) correlating a result of the calculation with an objective measurement of the symptom or sign to produce patient reported outcome information for assessing the health of the patient.

### Abbreviations:

CDC Centers for Disease Control
CFA Confirmatory Factor Analysis
CORECTS Colorectal Evaluation of Clinical Therapeutics Scale
CTT Classical Test Theory
EFA Exploratory Factor Analysis
FDA Food and Drug Administration
HD Hemorrhoid Disease
HD-SIM™ system Hemorrhoid Disease Symptom Impact Measure system
HIPAA Health Insurance Portability and Accountability Act
IRT Item Response Theory
NDA New Drug Application
PRO Patient-Reported Outcome
PROM Patient Reported Outcome Measure
PROMIS Patient-Reported Outcome Measurement Information System
QOL Quality of Life
WHO World Health Organization
HD-SIM™ is a trademark of Cristcot LLC.

### BRIEF DESCRIPTION OF THE DRAWINGS

FIG. 1 is a diagram showing a conceptual model of hemorrhoid disease, according to some embodiments.

FIG. 2 is graph of data relating to hemorrhoid disease, according to some embodiments.

FIGS. 3A-J are views presented in a patient reported outcome measure, according to some embodiments.

The foregoing will be apparent from the following more particular description of example embodiments, as illustrated in the Tables and drawings interspersed herein. The drawings are not necessarily to scale, emphasis instead being placed upon illustrating embodiments.

### DETAILED DESCRIPTION

A description of example embodiments follows.

Hemorrhoid disease (HD) affects 10 million people in the U.S. at any given time (Pullen, Luchtefeld), and 50% of the U.S. population (i.e., 167 million people) will develop symptomatic hemorrhoids during their lifetime. Up to 85% of women experience HD in the third trimester of pregnancy (Simmons). The prevalence of HD in the population is about 4%, and its prevalence increases with age. Over half of people over age 50 experience symptoms (Riss).

While hemorrhoids are present in all persons, they are generally unrecognized until they become symptomatic (Lunsford). Approximately 60% of people with HD experience symptoms (Sun). The most common symptoms of HD are pain, bleeding, itching, swelling and discomfort. HD patients also report bloating, abdominal pain, excessive straining, feeling of incomplete evacuation, and repeated toilet visits (Johannsson). HD symptoms negatively impact well-being, social life, and overall quality of life (Johannsson) but not mortality (Lunsford).

Symptoms develop when venous drainage of the rectum is increased, resulting in dilation of the sinusoids supplied by the superior and middle hemorrhoidal arteries and the superior rectal artery. Factors that increase pressure in the pelvic floor contribute to the formation of hemorrhoids, such as chronic frequent diarrhea, obesity, pregnancy and labor, anal intercourse, pelvic floor dysfunction, and decompensated cirrhosis complicated by ascites (Mott).

Treatment for HD may depend upon severity of symptoms or response failure to prior treatment interventions. Initial treatment of hemorrhoids often includes dietary and lifestyle changes (high fiber diet), warm water sitz baths alternating with cold compresses, use of stool softeners, non-steroidal anti-inflammatory drugs, and over-the-counter creams and pads (Lunsford). Conservative management is effective in many cases. In more severe cases, and particularly for internal hemorrhoids refractory to conservative therapy, more invasive approaches include rubber band ligation, infrared photocoagulation, sclerotherapy, and surgery. These more invasive approaches are painful and are associated with longer recovery times and higher complication rates. Symptoms of HD have variable rates of recurrence. For example, after banding, 30% to 50% of individuals will have recurrence of hemorrhoids within 5 to 10 years compared with 2% to 5% after operative hemorrhoidectomy (Mott).

The annual U.S. cost in 2014 was estimated to be a high of $2.4 billion (Yang). Given the substantial disease burden and cost, the time has come for targeted research to understand the cause, symptoms, and best treatment for people with symptomatic hemorrhoids (Sandler).

In order to meet this need, it is useful to have reliable and valid methods for assessing the severity of HD and its impact on patient QOL. Current research has documented that clinician-reported measures of HD severity are problematic and there currently doesn't exist a validated measure for the symptomatic impact of HD. For example, Dekker et al. performed a reliability study of the Goligher classification system (Goligher), documenting moderate inter-rater agreement at grades I and IV, and only fair agreement at grades II and III (Dekker).

Because the impact of HD is related to symptoms that are best assessed by the individual experiencing them, HD is a condition that would be well-served by having a validated patient-reported outcome (PRO) measure. A number of studies have reported on the development of PROs for HD. Longchamp et al. (Longchamp) did a systematic review of the HD literature to evaluate scoring systems, and concluded that there is an urgent need for a simple, externally validated and exhaustive scoring system which includes the Coloproctology Core Outcome Set for HD (Van Tol). This Core Outcome Set includes a PRO and clinician-assessed outcomes. All HD PROs are very similar in their reliance on a similar and short set of hemorrhoidal symptoms in their evaluation of disease severity. They do not address the multidimensional impact of HD on patient's lives and QOL. Further, none of these scales has been properly validated according to the 2009 Food and Drug Administration (FDA) Guidance for PROs (FDA). Accordingly, none of these scales can be used for gaining approval and labeling of an HD treatment.

### Methods Phase I: Interview Phase to Develop Conceptual Model and Items for HD PRO

### Endpoint Model

At project initiation, we anticipated that the PRO would include a Symptoms subscale, intended to be the primary endpoint for subsequent clinical trials for the measurement of disease-impact severity, and that it would also include subscales reflecting the impact of HD on QOL. These subscales could be secondary or exploratory endpoints for subsequent clinical trials.

### Initial Conceptual Model

The initial conceptual model was developed after considering work done by the European Society of Coloproctology to create a Core Outcome Set (Van Tol). This Delphi study queried healthcare professionals regarding outcome domains and outcomes deemed relevant to HD from a systematic literature review. The review of the previous work suggested that key domains were symptoms (primary outcome), complications, recurrence, and patient satisfaction (secondary outcomes).

Building on ideas within this Core Outcome Set and consistent with the World Health Organization (WHO) concept of health (WHO), we anticipated that the new PRO's conceptual model should be multidimensional, and should tap symptoms, physical functioning, mental health, and social functioning focused on content specifically relevant to HD.

Resulting Item Pool of the HD-SIM system

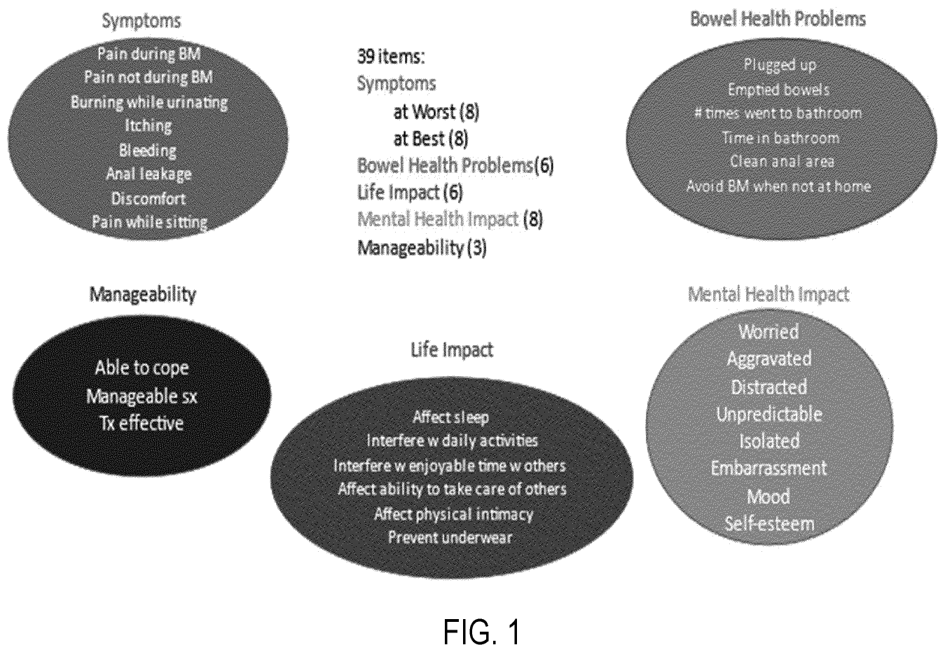

The 39 items in the final version of the item pool reflected a conceptual model comprised of five aspects of HD: symptoms (at best and at worst), bowel health, life impact, mental health impact, and manageability, as illustrated in FIG. 1. The symptoms items were asked “at its worst” and “at its best” in order to enable capturing symptom variability.

### Recall Period

The recall period for all HD-SIM system items is “within the past 24 hours,” which is reliable and valid regardless of whether the questions are asked daily or only at baseline, Midpoint Visit and End of Study.

### Response Options

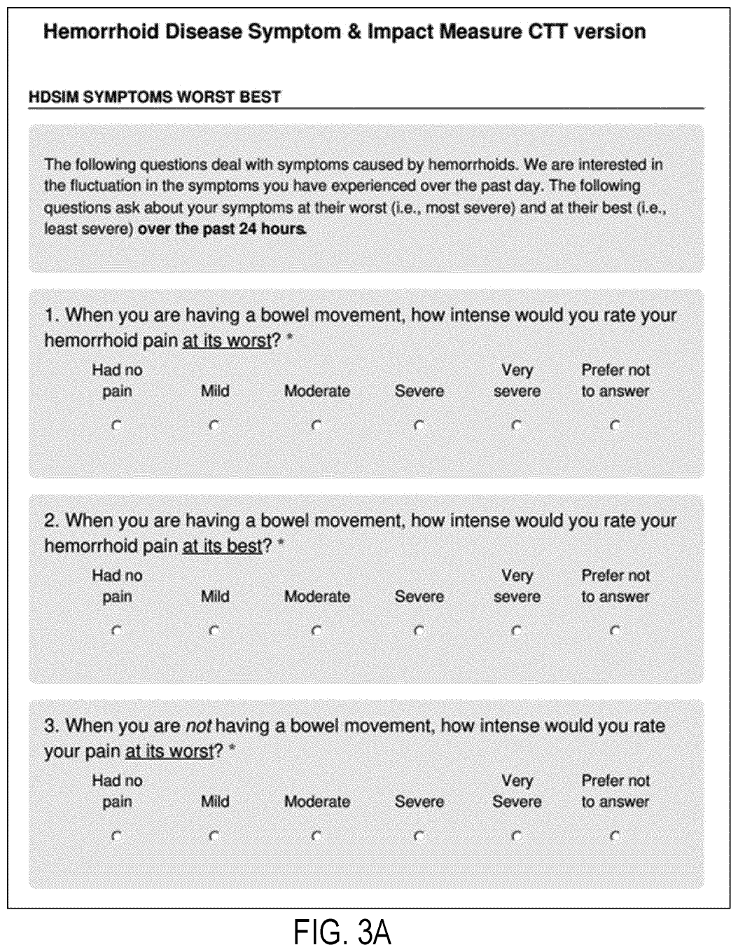

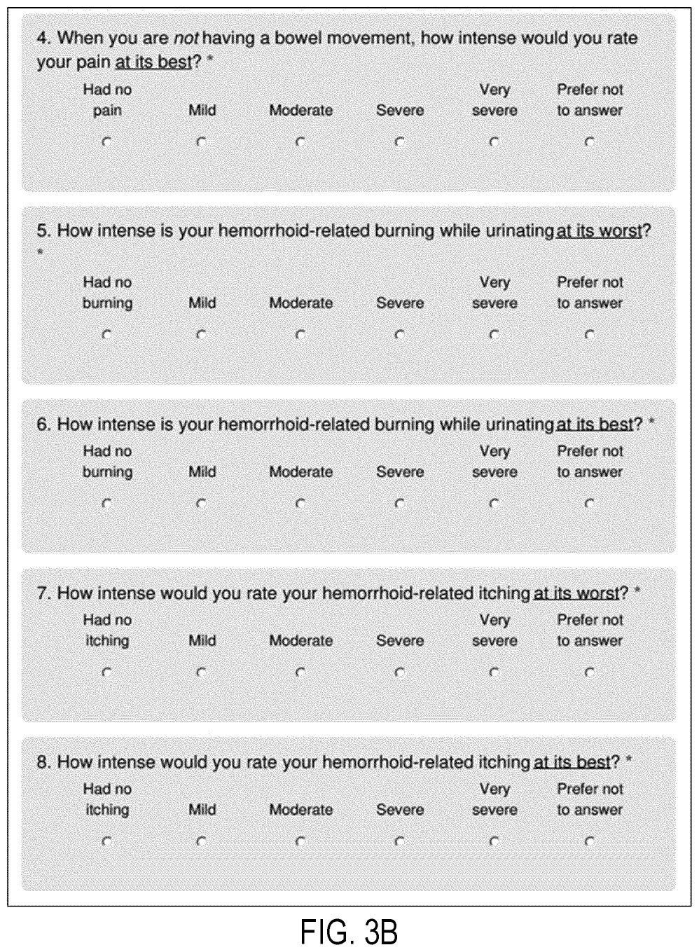

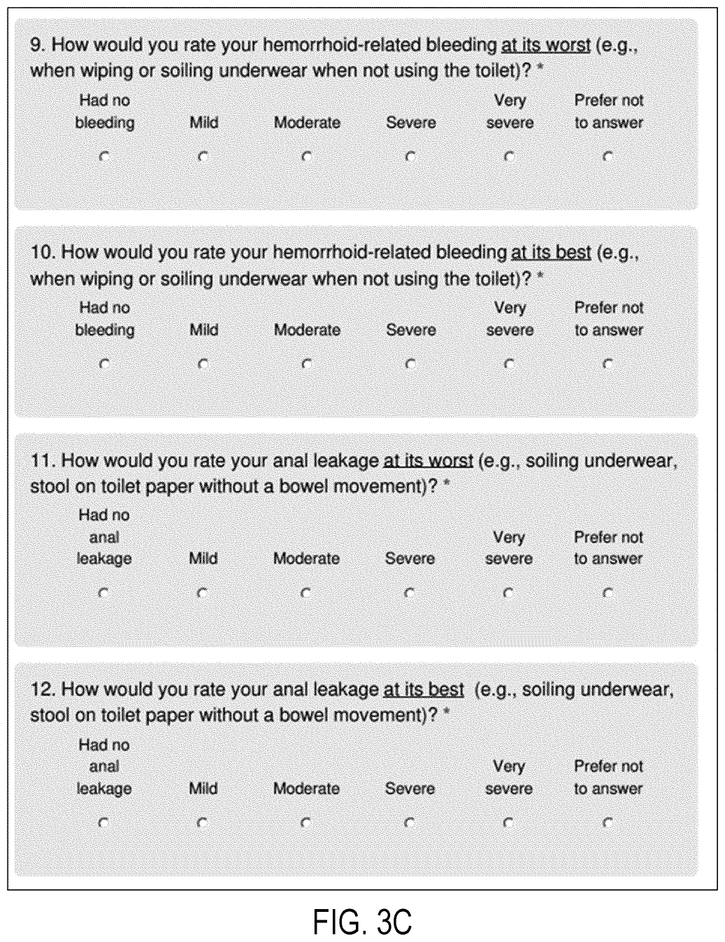

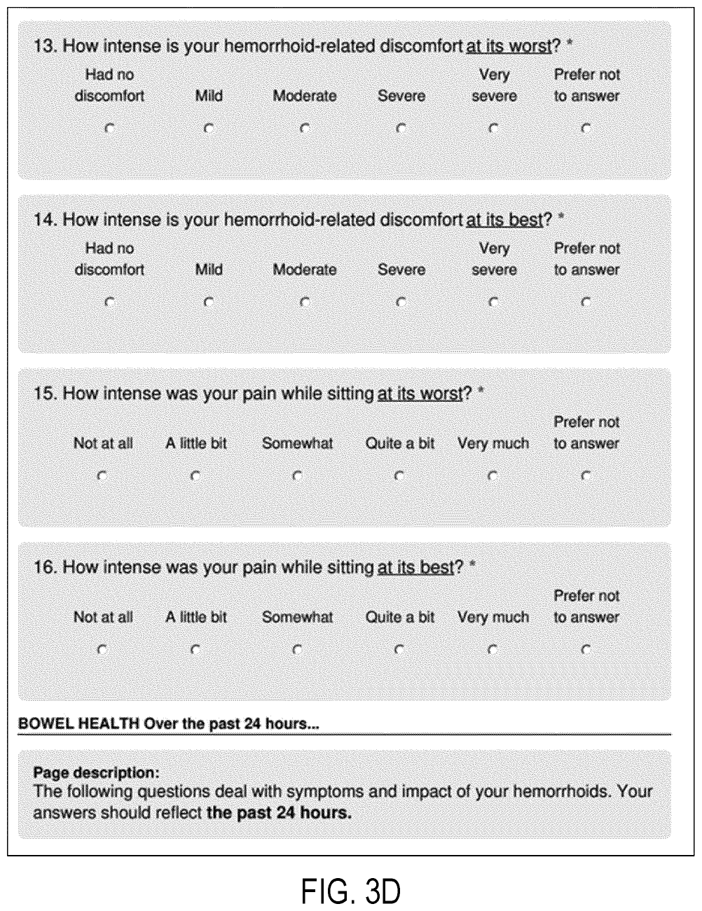

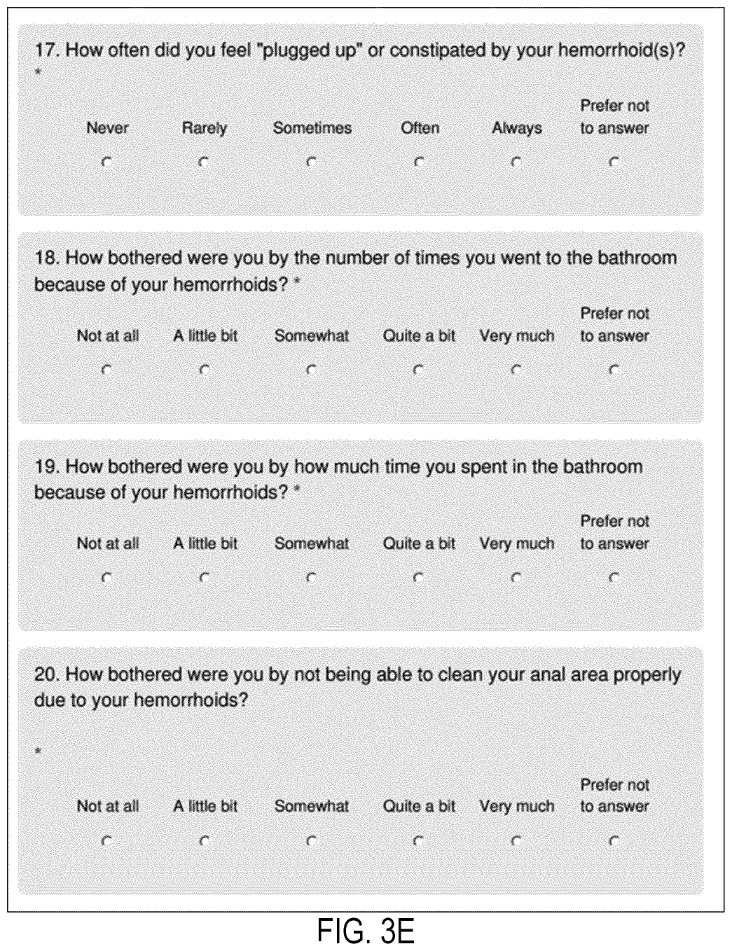

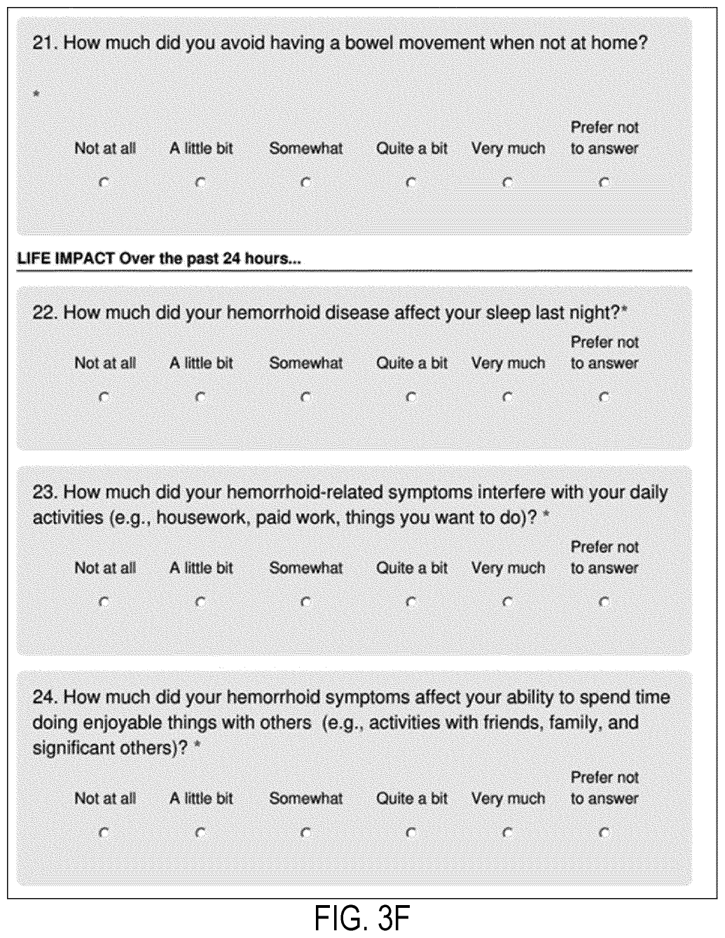

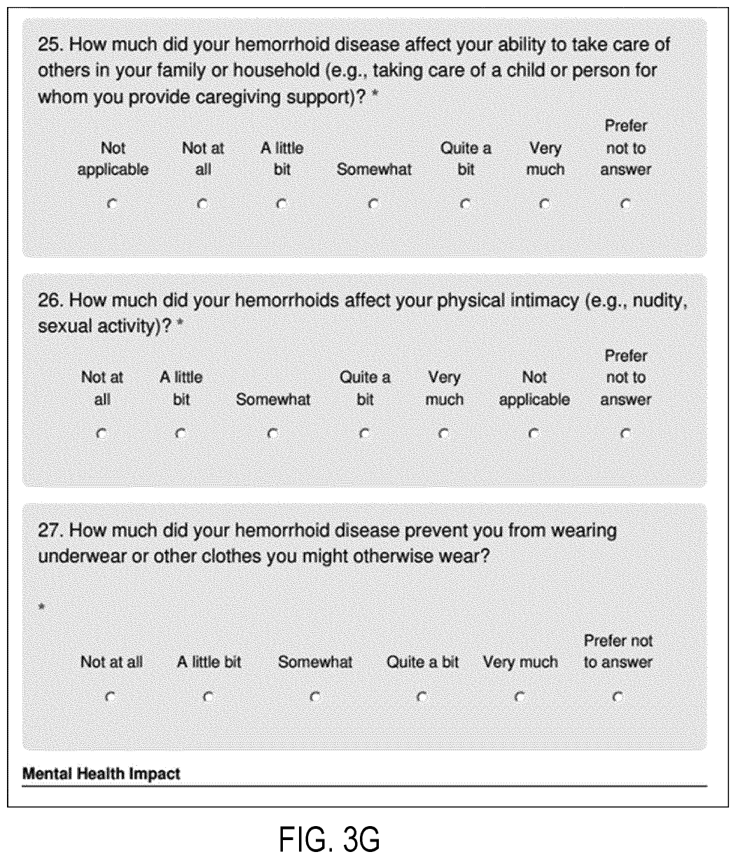

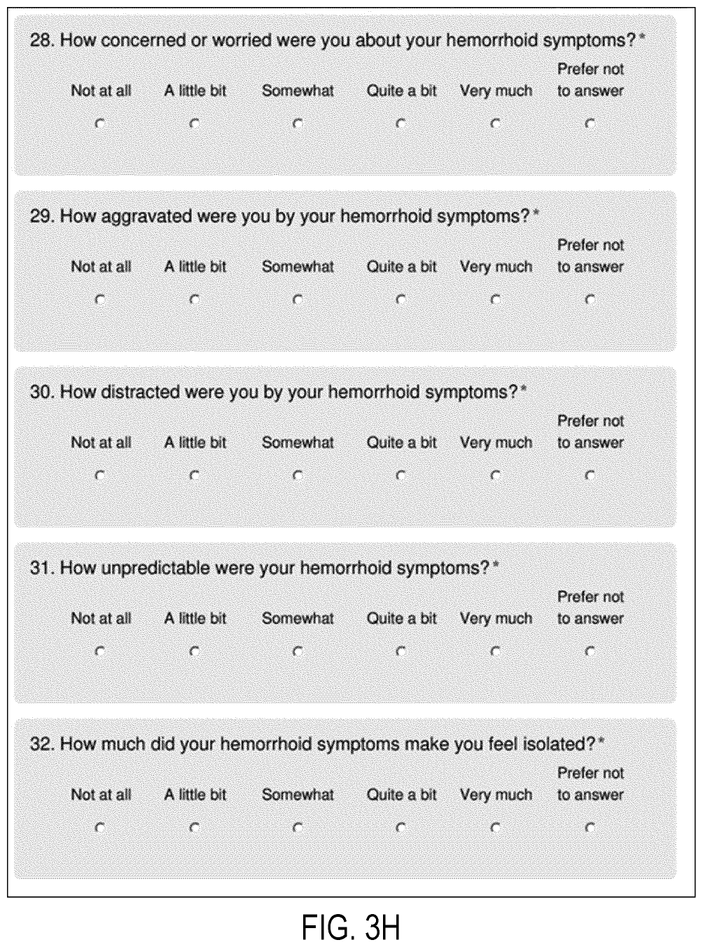

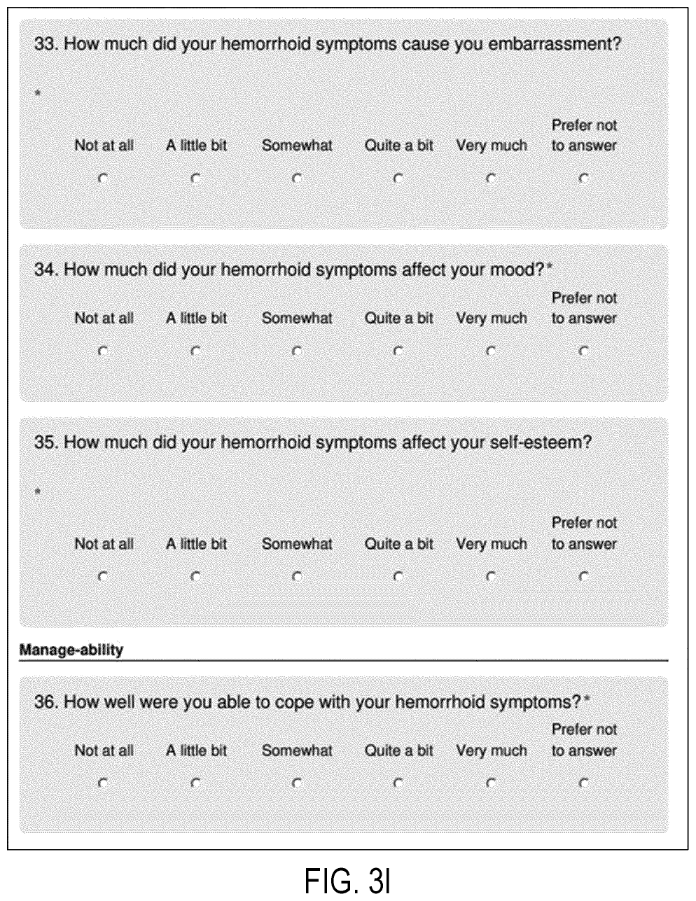

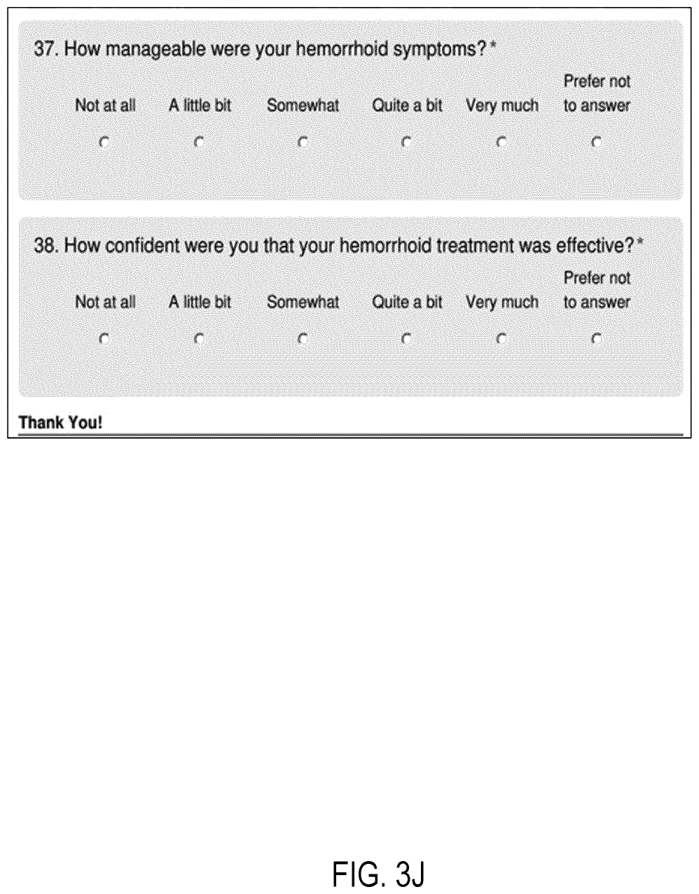

As visible in FIGS. 3A-J, the response options for the symptom items ranged from “had no [symptom]” to “very severe.” Included was an option for “prefer not to answer” in order to require each item to have a response in the online survey (i.e., participants had to provide an answer in order to click to the next item or page) without compelling individuals to answer if they were not comfortable doing so. This construct of response options complies with ethical and scientific guidelines.

As visible in FIGS. 3A-J, most other items of the HD-SIM system used response options ranging from “not at all” to “very much” or “prefer not to answer.” Response options were slightly different for an item that asked about how often a participant felt plugged up or constipated, with response options ranging from “never” to “always” or “prefer not to answer.”

### Study Design for Methods Phase II

Web-Based Survey. This study was administered using the web-based survey using the Health Insurance Portability and Accountability Act (HIPAA)-compliant, secure, web-based Alchemer engine (www.alchemer.com).

Design. The study was a cross-sectional study of about 1,000 individuals with HD, with a subset study of 100 randomly selected subjects to complete a retest within one to two weeks of the original survey completion. The HD baseline and retest samples were stratified by self-reported HD symptom severity (i.e., mild, moderate, severe). Study participants were sampled to be nationally representative of the United States population for stratification of age and race.

### Results Phase II-Quantitative Item-Development Phase

### Sample

Data were collected on 1,075 individuals. Data from 9 individuals were removed from the analytic data set because they did not provide a viable answer to the question about duration of HD (e.g., they answered “0” years), making their HD status questionable. Table 3 provides descriptive characteristics of the Phase II study sample.

Demographic Characteristics. Participants had a mean age of 47.5. The sample included 639 females, 424 males, and 1 transgender individual. Seventy-one percent of the individuals reported White race, 21% Black, and the remainder endorsed other race categories. Fifteen percent endorsed Hispanic ethnicity. Most of the sample (57%) was married or cohabiting; most lived with a spouse or partner (58%) and/or other relative (48%); and 16% lived alone. About a quarter of the sample had attained an education level ranging from grade school to high school or vocational degree; 57% had some college to a completed 4-year university degree; and 19% of the sample had attained a graduate degree. Over half of the sample was currently working. Forty-five percent of the sample endorsed difficulty paying bills (somewhat to extremely).

Health and HD-specific Characteristics. Most of the sample (64%) were non-smokers, and 87% of the sample had health insurance. They reported an average of 3 comorbidities, and an average duration of HD of 12.2 years. About a third of the sample reported mild HD; 45% reported moderate; and 23% reported severe HD. About two-thirds of the sample reported having internal hemorrhoids, and about ¾ reported external (participants could check both types).

### Scoring of Items and Domains

Two sets of scores were created. The CTT scores were computed for the 6 conceptually driven subscales, while IRT scores were computed for the 2 subscales derived primarily on model fit-based statistics. Specifically, the CTT-based scores were computed as the mean of items within each subscale, as long as the participant provided a response for 50% of the items (i.e., at least 4 items must be answered on an 8-item subscale). The IRT-based scores were derived from the raw summed score to IRT scale score conversion table based on the item parameters obtained in the abovementioned analyses. The IRT scale scores are T-scores with a mean of 50 and a standard deviation of 10.

### Comparison of Performance

A series of analyses were then implemented to evaluate the two sets of scores according to performance on important PRO metrics.

Overlap. Results of Pearson correlation analysis revealed that the two sets of scores were highly correlated. The Symptoms at Worst CTT score correlated 0.97 with the Symptoms at Worst IRT score. The Bowel Health Problems, Life Impact, and Mental Health CTT scores correlated highly with the Overall Impact IRT score (r=0.91, 0.90, and 0.97, respectively). All of these correlation coefficients were large effects sizes using Cohen's criteria (38). Thus, the overlap between the two sets of scores was very high.

Cross-sectional Construct validity

### Discriminating Known Groups

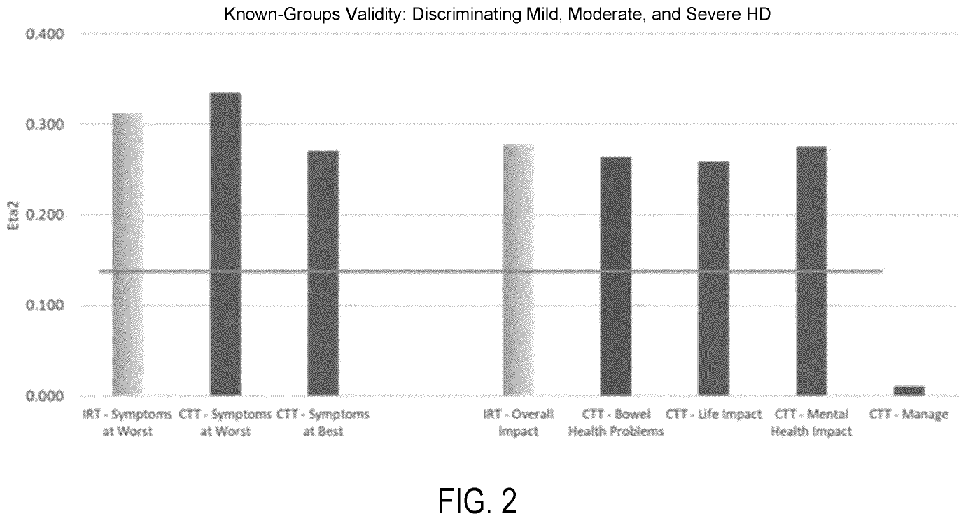

FIG. 2 displays results of analysis of variance models investigating how much each subscale explained variance in discriminating mild, moderate, and severe HD groups. All subscales but the CTT Manageability demonstrated large Effect Sizes(ES) in detecting differences among HD severity groups (bars exceeding orange cut point are large effect sizes).

### Convergent and Divergent Validity

Supplementary Table 5 provides the descriptive statistics on the generic QOL measures and the HD-specific PROs.

### Respondent Burden

The HD-SIM system was tested for respondent burden using the Alchemer survey engine's test function. It generated low fatigue scores and high accessibility scores. The HD-SIM system in its entirety was estimated to take 4 minutes to complete, and the Symptoms at Worst and Symptoms at Best subscales were estimated to take 2 minutes to complete.

The Supplementary Tables below illustrate an embodiment of an HD-SIM system as applied to a hemorrhoidal study and analysis (e.g., via responses to the questions shown in FIGS. 3A-J). Data collected and analyzed would be different for a different illness, injury, or condition.

SUPPLEMENTARY TABLE 1
Results of the Cognitive Interviews
Supplementary Table 1. Results of Cognitive Interviews

| | Round One | Round Two | Round Three | Round Four | Overall |
|---|---|---|---|---|---|
| No. Patients | 3 | 5 | 5 | 3 | 16 |
| Duration of HD (years) | Not asked | 1-2* | 4-15 | 5-30 | 1-30 |
| HD Severity | | | | | |
| Mild | 0 | 1 | 2 | 0 | 3 |
| Moderate | 3 | 4 | 3 | 2 | 12 |
| Severe | 0 | 0 | 0 | 1 | 1 |
| Gender | | | | | |
| Females | 3 | 3 | 3 | 3 | 12 |
| Males | 0 | 1 | 2 | 0 | 3 |
| Non-Binary | 0 | 1 | 0 | 0 | 1 |
| Age Range (years) | 33-39 | 23-55 | 36-45 | 31-61 | 23-61 |
| Marital status | | | | | |
| Never Married | 2 | 2 | 2 | 1 | 7 |
| Married/Cohabiting | 1 | 1 | 3 | 2 | 7 |
| Divorced | 0 | 2 | 0 | 0 | 2 |
| Declined to answer | 0 | 0 | 1 | 0 | 1 |
| Hispanic ethnicity | 1 | 0 | 0 | 0 | 1 |
| Race | | | | | |
| White | 3 | 5 | 4 | 1 | 13 |
| Black | 0 | 0 | 0 | 1 | 1 |
| South Asian | 0 | 0 | 1 | 0 | 1 |
| Education | | | | | |
| high school diploma | 0 | 1 | 1 | 1 | 3 |
| some college | 2 | 3 | 2 | 0 | 7 |
| technical vocation degree | 1 | 0 | 0 | 0 | 1 |
| 4-year university degree | 0 | 0 | 0 | 1 | 1 |
| masters degree | 0 | 1 | 2 | 1 | 4 |
| No. Items Tested | 16 | 30 | 32 | 37 | 37 |
| No. items reflecting symptoms | 5 at worst, | 7 at worst, | 7 at worst, | 7 at worst, | 7 at worst, |
| | 5 at best | 7 at best | 7 at best | 7 at best | 7 at best |
| No. items reflecting impact | 4 | 12 | 18 | 23 | 23 |
| Suggested changes to existing items | | | | | |
| % no change suggested | 63% | 75% | 85% | 87% | — |
| % wordsmithing suggested | 38% | 20% | 5% | 6% | — |
| % item not relevant to interviewee | 0 | 0 | 5% | 4% | — |
| but knows it is for other people | | | | | |
| % drop-item suggested | 0 | 5% | 8% | 4% | — |
| No. new items (content) suggested | 6 | 10 | 5 | 0 | — |
| Instructions | Specified | Underline “at | Added text to | — | — |
| | interest in | its worst” and | explain that we are | | |
| | symptoms | “at its best” and | interested in the | | |
| | over the past | bold “in the | fluctuation of | | |
| | 24 hours. | past 24 hours” | symptoms the past | | |
| | | | day. Added short, | | |
| | | | distinct instruction | | |
| | | | for impact items. | | |
| Response Options | None at all- | Had no | (No changes | (No changes | — |
| | Most severe | (specific | made) | made) | |
| | | symptom)- | | | |
| | | Very severe | | | |
| | | Never-Always | | | |
| | | Not at all- | | | |
| | | Very much | | | |
| Suggested changes | Anchor | Add “Do not | Bigger font | Added “Not | Added “Prefer not |
| | words for | know/Prefer | “in the | applicable” | to answer” to all |
| | each response | not to answer” | past 24 hours” | to physical | items and removed |
| | option | and bold “in | | intimacy | “Not applicable” |
| | | the past 24 | | item | from physical |
| | | hours” | | | intimacy item |

*Questions added mid-round

SUPPLEMENTARY TABLE 2
List of measures for Web-based Validation Study
Supplementary Table 2. List of measures for Web-based Validation Study

| | |
|---|---|
| Purpose | Measure |

Construct Validation

| | |
|---|---|
| HD-specific measures | Convergent validity: highest correlation coefficients |
| | hypothesized |
| | ColoRectal Evaluation of Clinical Therapeutics Scale |
| | (CORECT) |
| | Sodergren Scale |
| | Hemorrhoidal Disease Symptom Score, Short Health ScaleHD |
| | HD Symptom Impact Measure (HDSIM) |
| Cross-section Construct Validity | Convergent validity: moderate correlation coefficients |
| | hypothesized |
| | PROMIS-29 |
| | Divergent validity: lowest correlation coefficients |
| | hypothesized |
| | Ryff Environment Mastery, NeuroQOL Positive Affect & |
| | Well-Being, DeltaQuest Wellness, Patient Experience with |
| | Treatment and Self-Management Medical Information |
| | subscale, Life Stress Questionnaire, DeltaQuest Reserve- |
| | Building Activities Current Social Support subscale |
| Known-groups validity | HD severity |
| Longitudinal Construct Validity | Stability versus responsiveness |
| | Global Assessment of Change item (at retest follow-up) for |
| | subgrouping patients as Supplementary Table, worsening, or |
| | improving |

Descriptive Variables

| | |
|---|---|
| Demographics | year of birth, gender, race, ethnicity, marital status, with whom |
| | live |
| | difficulty paying bills, education, employment status, smoking |
| | status, health insurance, comorbidities |

SUPPLEMENTARY TABLE 3
Validation Study Sample Characteristics (n = 1066)
Supplementary Table 3. Sample Characteristics (n = 1066)

| Variable | Mean | SD |
|---|---|---|
| Age | 47.5 | (15.1) |
| range | 22-94 | |
| HD Duration in years | 12.2 | 12.9 |
| range | .3-70 | |
| No. Comorbidities | 3.12 | (2.4) |
| range | 0-15 | |
| | N | % |
| HD Severity at baseline | | |
| mild | 343 | 32.2 |
| moderate | 477 | 44.7 |
| severe | 246 | 23.1 |
| Type of Hemorrhoid* | | |
| Internal | 689 | 64.6 |
| External | 775 | 72.7 |
| Gender | | |
| Male | 424 | 39.8 |
| Female | 639 | 59.9 |
| Transgender | 1 | 0.1 |
| Race | | |
| White | 757 | 71 |
| Black | 220 | 20.6 |
| Middle Eastern | 10 | 0.9 |
| South Asian | 27 | 2.5 |
| Other Asian | 50 | 4.7 |
| Amer. Indian or Alaskan Native | 36 | 3.4 |
| Native Hawaiian or Pacific Islander | 3 | 0.3 |
| Other Asian | 33 | 3.1 |
| NA/Prefer not to answer | 2 | 0.2 |
| Hispanic Ethnicity | 164 | 15.4 |
| Marital | | |
| Never Married | 256 | 24.0 |
| Married or Cohabiting | 607 | 56.9 |
| Separated or Divorced | 145 | 13.6 |
| Widowed | 56 | 5.3 |
| NA/Prefer not to answer | 2 | 0.2 |
| With whom live | | |
| Spouse/Partner | 621 | 58.3 |
| Other Relative | 510 | 47.8 |
| Friend/Companion | 48 | 4.5 |
| Pet(s) | 293 | 27.5 |
| Alone | 168 | 15.8 |
| Domestic/Health Related Help | 2 | 0.2 |
| Nursing or Sheltered Home | 1 | 0.1 |
| NA/Prefer not to answer | 19 | 1.8 |
| Difficulty Paying Bills | | |
| Not difficult at all | 355 | 33.3 |
| Slightly difficult | 206 | 19.3 |
| Somewhat difficult | 193 | 18.1 |
| Very difficult | 141 | 13.2 |
| Extremely difficult | 146 | 13.7 |
| NA/Prefer not to answer | 25 | 2.3 |
| Education | | |
| Less than 12th grade | 22 | 2.1 |
| High school diploma | 180 | 16.9 |
| Some college | 208 | 19.5 |
| Technical (Vocational) degree | 57 | 5.3 |
| 2-year University degree | 117 | 11.0 |
| 4-year University degree | 280 | 26.3 |
| Masters degree | 163 | 15.3 |
| Doctoral degree | 38 | 3.6 |
| NA/Prefer not to answer | 1 | 0.1 |
| Employment Status | | |
| Currently working | 599 | 56.2 |
| Unemployed | 152 | 14.3 |
| Retired | 191 | 17.9 |
| Disabled | 105 | 9.8 |
| Prefer not to answer | 19 | 1.8 |
| Smoking/Vaping | | |
| No, never | 482 | 45.2 |
| Yes, only in the past | 200 | 18.8 |
| Yes, some days currently | 106 | 9.9 |
| Yes, every day currently | 254 | 23.8 |
| Prefer not to answer | 24 | 2.3 |
| Has health insurance | | |
| No | 117 | 11.0 |
| Yes | 929 | 87.1 |
| Prefer not to answer | 20 | 1.9 |

*Participant could check all that apply
**“Training” is brief term to reflect training, amount of complex decision-making, adapting to changing circumstances, or flexible response to different types of people.

SUPPLEMENTARY TABLE 4
Factor Analysis Results for CTT-based Scales
Supplementary Table 4. Factor Analysis Results for CTT-based Scales

| | | | Alpha |
|---|---|---|---|
| | Factor | Explained | Reliability |
| Subscale and Items | Loading | Variance | Coefficient |
| Symptoms at Worst CTT score | | 65% | 0.94 |
| How intense is your hemorrhoid-related discomfort at its worst? | 0.84 | | |
| When you are having a bowel movement, how intense would you rate your hemorrhoid | 0.84 | | |
| pain at its worst? | | | |
| When you are not having a bowel movement, how intense would you rate your pain at | 0.84 | | |
| its worst? | | | |
| How intense was your pain while sitting at its worst? | 083 | | |
| How would you rate your hemorrhoid-related bleeding at its worst (e.g., when wiping or | 0.80 | | |
| soiling underwear when not using the toilet)? | | | |
| How intense would you rate your hemorrhoid-related itching at its worst? | 0.79 | | |
| How intense is your hemorrhoid-related burning while urinating at its worst? | 0.78 | | |
| How would you rate your anal leakage at its worst (e.g., soiling underwear, stool on | 0.74 | | |
| toilet paper without a bowel movement)? | | | |
| Symptoms at Best CTT score | | 74% | 0.96 |
| How intense is your hemorrhoid-related discomfort at its best? | 0.89 | | |
| How intense is your hemorrhoid-related burning while urinating at its best? | 0.88 | | |
| How intense would you rate your hemorrhoid-related itching at its best? | 0.87 | | |
| When you are not having a bowel movement, how intense would you rate your pain at | 0.87 | | |
| its best? | | | |
| How intense was your pain while sitting at its best? | 0.86 | | |
| How would you rate your anal leakage at its best (e.g., soiling underwear, stool on toilet | 0.85 | | |
| paper without a bowel movement?) | | | |
| How would you rate your hemorrhoid-related bleeding at its best (e.g., when wiping or | 0.84 | | |
| soiling underwear when not using the toilet?) | | | |
| When you are having a bowel movement, how intense would you rate your hemorrhoid | 0.80 | | |
| pain at its best? | | | |
| Bowel Health CTT score | | 66% | 0.90 |
| How bothered were you by how much time you spent in the bathroom because of your | 0.90 | | |
| hemorrhoids? | | | |
| How bothered were you by the number of times you went to the bathroom because of | 0.88 | | |
| your hemorrhoids? | | | |
| How bothered were you by not being able to clean your anal area property due to your | 0.78 | | |
| hemorrhoids? | | | |
| How often did you feel plugged up or constipated by your hemorrhoid(s)? | 0.77 | | |
| How much did you avoid having a bowel movement when not at home? | 0.71 | | |
| Life Impact CTT score | | 72% | 0.94 |
| How much did your hemorrhoid symptoms affect your ability to spend time doing | 0.92 | | |
| enjoyable things with others (e.g., activities with friends, family, and significant others)? | | | |
| How much did your hemorrhoid-related symptoms interfere with your daily activities | 0.91 | | |
| (e.g., housework, paid work, things you want to do)? | | | |
| How much did your hemorrhoid disease affect your sleep last night? | 0.86 | | |
| How much did your hemorrhoid disease affect your ability to take care of others in your | 0.85 | | |
| family or household? recoded so that Not Applicable responses are Not at All | | | |
| How much did your hemorrhoid disease prevent you from wearing underwear or other | 0.85 | | |
| clothes you might otherwise wear? | | | |
| How much did your hemorrhoids affect your physical intimacy (e.g., nudity, sexual | 0.70 | | |
| activity)?* | | | |
| Mental Health Impact CTT score | | 75% | 0.96 |
| How distracted were you by your hemorrhoid symptoms? | 0.90 | | |
| How much did your hemorrhoid symptoms affect your mood? | 0.90 | | |
| How much did your hemorrhoid symptoms cause you embarrassment? | 0.89 | | |
| How much did your hemorrhoid symptoms make you feel isolated? | 0.88 | | |
| How much did your hemorrhoid symptoms affect your self-esteem? | 0.86 | | |
| How concerned or worried were you about your hemorrhoid symptoms? | 0.86 | | |
| How aggravated were you by your hemorrhoid symptoms? | 0.85 | | |
| How unpredictable were your hemorrhoid symptoms? | 0.77 | | |
| Manageability CTT score | | 62% | 0.82 |
| How manageable were your hemorrhoid symptoms? | 0.93 | | |
| How well were you able to cope with your hemorrhoid symptoms? | 0.73 | | |
| How confident were you that your hemorrhoid treatment was effective? | 0.69 | | |

*Recoded so that Not Applicable responses are Not at All

SUPPLEMENTARY TABLE 5
Descriptive Statistics of Generic and HD-Specific PROs
Supplementary Table 5. Descriptive Statistics of Generic and HD-Specific PROs

| | | | | |
|---|---|---|---|---|
| N | Mean | SD | Minimum | Maximum |

Generic QOL Scores

| | | | | | |
|---|---|---|---|---|---|
| PROMIS29 Anxiety/Fear | 1054 | 60.24 | 11.52 | 40.30 | 81.40 |
| PROMIS29 Depression/Sadness | 1053 | 57.44 | 11.61 | 41.00 | 79.30 |
| PROMIS29 Fatigue | 1054 | 56.94 | 11.42 | 33.70 | 75.80 |
| PROMIS29 Pain Interference | 1052 | 57.84 | 9.75 | 42.10 | 75.00 |
| PROMIS29 Physical Function | 1056 | 43.58 | 9.40 | 22.60 | 57.00 |
| PROMIS29 Sleep Disturbance | 1057 | 53.78 | 6.17 | 32.00 | 73.30 |
| NeuroQOL Positive Affect | 1055 | 51.30 | 8.26 | 26.30 | 68.00 |
| DeltaQuest Wellness | 1018 | 0.02 | 0.87 | −2.45 | 2.41 |
| Ryff Environmental Mastery | 1066 | 27.58 | 20.14 | −177.00 | 230.00 |
| Social Support | 1045 | 17.44 | 6.15 | 5.00 | 26.00 |
| PETS Literacy Score | 982 | 30.59 | 21.37 | 0.00 | 100.00 |

HD-Specific Scores

| | | | | | |
|---|---|---|---|---|---|
| CORECTS Summary Score | 1058 | 32.79 | 14.71 | 0.00 | 60.00 |
| Sodergren Summary Score | 1058 | 7.25 | 4.61 | 0.00 | 16.00 |
| HDSS Summary Score | 1053 | 11.68 | 4.50 | 0.00 | 20.00 |
| SHSHD Summary Score | 1053 | 12.75 | 4.71 | 3.00 | 21.00 |
| Worst symptoms CTT score | 1051 | 2.91 | 1.06 | 1.00 | 5.00 |
| Best symptoms CTT score | 1051 | 2.44 | 1.13 | 1.00 | 5.00 |
| Bowel problems CTT score | 1051 | 3.14 | 1.14 | 1.00 | 5.00 |
| Life impact problems CTT score | 1050 | 2.56 | 1.27 | 1.00 | 5.00 |
| Mental health impact CTT score | 1052 | 2.99 | 1.23 | 1.00 | 5.00 |
| Manage condition CTT score | 1050 | 3.36 | 1.02 | 1.00 | 5.00 |

### Recommendations for Use in Clinical Trials

Frequency of Assessments. We recommend using the Symptoms at Worst and Symptoms at Best as a daily-diary assessment prior to, during, and after treatment with an Investigational Product intended to treat HD. In contrast, we recommend using the impact subscales at the beginning, midpoint and end of the trial. This measurement approach is parsimonious, will provide a useful measurement of symptom severity and variability, and will document the treatment effect on important life domains.

### Study Limitations

The present study for the development and validation of the HD-SIM system has many advantages, including a robust sample size which is relatively evenly distributed by HD severity. The study sample is diverse with respect to race and ethnicity and reflects the general United States population in terms of age, gender, and region. The only study limitation is that this is the first study using the HD-SIM system. Since measures are validated in an iterative manner over many studies and over time, the present study results should be considered preliminary.

### CONCLUSIONS

The HD-SIM system is a reliable and valid PRO for use in HD clinical trials and observational clinical research. Its accessible, brief format provides a useful tool that has demonstrated reliability and validity, both cross-sectionally and longitudinally. The HD-SIM system is overall ready and fit-for-purpose for HD studies as a measurement of symptom impact of a treatment intervention.

### Exemplifications

### Computer-Implemented Embodiment

The HD-SIM system may be implemented in an individual electronic device, via a network system with edge nodes and a central server, or other computer system or network configuration known in the art configurable for supporting the functions disclosed herein. For example, the HD-SIM system may provide a portal through which a patient may be presented an HD-SIM system questionnaire, as provided below for a hemorrhoidal condition. The user may select answers in the form of selecting radio buttons or other graphical user interface element to capture input from the patient. The answers may be quantified for mathematical processing to make assessment(s) in accordance with embodiments disclosed herein. A server supporting the portal may then perform operations to produce the output as described hereinabove.

### Supplemental Data Embodiment

HD-SIM system may enable submission of physical measurements in addition to patient-provided data. For example, for a hemorrhoidal disease application, at least one physical measurement may include a measurement of blood flow through the hemorrhoidal vein or artery. Alternatively or in addition, the at least one physical measurement may be made to quantify size, shape, and volume of a hemorrhoidal vein or artery, or other relevant structure, at the beginning and end of a study period.

Tools that may be employed for such physical measurements may be a high resolution anoscopy camera and a hemorrhoid treatment Doppler system. The Doppler measurement is used to provide a model of varicosity in an afflicted region, and specifically in relation to varicosity of not only hemorrhoid veins but other areas of the body. A ‘4th dimension’ of measurement of pressure and flow in the vein (or artery) can be measured and quantified over the measurement of length, width, and volume. Use of devices to perform such measurements in a clinical study protocol, as determined by a central reading (not the treating physician) to offer a measurement at a baseline (i.e., beginning) and at an end of treatment, may allow an examination of an associative property of Patient Reported Impact to any change or stasis of a quantifiable measurement by the controlled protocol and reading of the measurement of the anatomy.

### REFERENCES

1. Pullen Jr RL. Hemorrhoidal disease: What nurses need to know. Nursing2022. 2022;52 (5): 19-24.
2. Simmons S. Ano-rectal disorders in pregnancy. BJOG: An International Journal of Obstetrics & Gynaecology. 1964;71 (6): 960-962.
3. Riss S, Weiser FA, Schwameis K, et al. The prevalence of hemorrhoids in adults. International Journal of Colorectal Disease. 2012; 27 (2): 215-220.
4. Lunsford TN, Atia MA, Kagbo-Kue S, Harris LA. A Pain in the Butt: Hemorrhoids, Fissures, Fistulas, and Other Anorectal Syndromes. Gastroenterology Clinics. 2022;51 (1): 123-144.
5. Sun Z, Migaly J. Review of hemorrhoid disease: presentation and management. Clinics in Colon and Rectal Surgery. 2016; 29 (01): 022-029.
6. Johannsson HÖ, Graf W, Påhlman L. Bowel habits in hemorrhoid patients and normal subjects. American Journal of Gastroenterology. 2005;100 (2): 401-406.
7. Yang JY, Peery AF, Lund JL, Pate V, Sandler RS. Burden and cost of outpatient hemorrhoids in the United States employer-insured population, 2014. The American Journal of Gastroenterology. 2019; 114 (5): 798.
8. Sandler RS, Peery AF. Rethinking what we know about hemorrhoids. Clinical Gastroenterology and Hepatology. 2019; 17 (1): 8-15.
9. Goligher JC. Surgery of the Anus, Rectum and Colon. 4th ed. Bailliere Tindall. 1980:924-925.
10. Dekker L, Han-Geurts I, Grossi U, Gallo G, Veldkamp R. Is the Goligher classification a valid tool in clinical practice and research for hemorrhoidal disease? Techniques in Coloproctology. 2022:1-6.
11. Van Tol R, Kimman M, Melenhorst J, et al. European Society of Coloproctology Core Outcome Set for haemorrhoidal disease: an international Delphi study among healthcare professionals. Colorectal Disease. 2019; 21 (5): 570-580.
12. Guidance for industry: patient-reported outcome measures: use in medical product development to support labeling claims (US Department of Health and Human Services Food and Drug Administration) (2009).
13. Kirwan JR, De Wit M, Frank L, et al. Emerging guidelines for patient engagement in research. Value in Health. 2017;20 (3): 481-486.
14. Ebrahimi N, Vohra-Miller S, Koren G. Anorectal symptom management in pregnancy: development of a severity scale. Journal of Population Therapeutics and Clinical Pharmacology. 2011; 18 (1): e99-e105.
15. Pucher P, Qurashi M, Howell AM, et al. Development and validation of a symptom-based severity score for haemorrhoidal disease: the Sodergren score. Colorectal Disease. 2015;17 (7): 612-618.
16. Rørvik HD, Styr K, Ilum L, et al. Hemorrhoidal disease symptom score and short health ScaleHD: new tools to evaluate symptoms and health-related quality of life in hemorrhoidal disease. Diseases of the Colon & Rectum. 2019; 62 (3): 333-342.
17. Cella D, Yount S, Rothrock N, et al. The Patient-Reported Outcomes Measurement Information System (PROMIS): progress of an NIH Roadmap cooperative group during its first two years. Medical care. 2007; 45 (5 Suppl 1): S3.
18. Ryff CD. Happiness is everything, or is it? Explorations on the meaning of psychological well-being. Journal of Personality and Social Psychology. 1989; 57:1069-1081.
19. Salsman JM, Victorson D, Choi SW, et al. Development and validation of the positive affect and well-being scale for the neurology quality of life (Neuro-QOL) measurement system. Quality of Life Research. 2013; 22 (9): 2569-2580.
20. Schwartz CE, Stucky BD, Stark RB. Expanding the purview of wellness indicators: validating a new measure that includes attitudes, behaviors, and perspectives. Health Psychology and Behavioral Medicine. 2021; 9 (1): 1031-1052.
21. Kickbusch I, Pelikan JM, Apfel F, Tsouros A. Health Literacy. WHO Regional Office for Europe; 2013.
22. Schwartz CE, Michael W, Zhang J, Rapkin BD, Sprangers MAG. Assessing reserve-building pursuits and person characteristics: Psychometric validation of the DeltaQuest Reserve-Building Measure. Quality of Life Research. 2018; 27:423-436. doi: 10.1007/s11136-017-1694-2
23. Krause NM, Jay GM. What do global self-rated health items measure? Med Care. 1994;32 (9): 930-942.

24 Wyrwich KW, Tardino VMS. Understanding global transition assessments. Quality of Life Research. 2006; 15 (6): 995-1004.
25. Hu Lt, Bentler PM. Cutoff criteria for fit indexes in covariance structure analysis: Conventional criteria versus new alternatives. Structural Equation Modeling. 1999;6 (1): 1-55.
26. Cohen J. A power primer. Psychological Bulletin. 1992; 112:155-159.
27. IBM SPSS Statistics for Windows. Version 28. IBM Corp; 2021.
28. Muthén LK, Muthén BO. Mplus User's Guide. Eighth ed. Muthén & Muthen; 1998-2022.
29. IRTPRO™ 6.0.6.0. Vector Psychometric Group, LLC; 2023.

### Example Embodiments and Incorporation by Reference

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A method of assessing health of a patient suffering from hemorrhoid disease, the method comprising:
a) presenting a patient reported outcome measure (PROM) for at least partial completion by the patient about hemorrhoid disease, the PROM including questions extending beyond a question about a symptom or sign of hemorrhoid disease; and
b) performing a calculation as a function of responses to the questions to assess the health of the patient.

2. The method according to claim 1 further comprising correlating a result of the calculation with an objective measurement of the symptom or sign to produce patient reported outcome information for assessing the health of the patient.

3. The method according to claim 1 wherein the questions include at least a subset of the following related to corresponding questions:
a) symptoms: pain during bowel movement (BM), pain not during BM, burning while urinating, itching, bleeding, anal leakage, discomfort, pain while sitting;
b) bowel health: plugged up, emptied bowels, number of times went to the bathroom, time in bathroom, clean anal area, and avoid BM while not at home;
c) manageability: able to cope, manageable symptom effect, treatment effect effective;
d) mental health impact: worried, aggravated, distracted, unpredictable, isolated, embarrassment, mood, self-esteem;
e) life impact: affect sleep, interfere with daily activities, interfere with enjoyable time with others, affect ability to take care of others, affect physical intimacy, and protect underwear.

4. The method according to claim 3 wherein the questions include a question about whether burning while urinating is experienced or whether pain occurs during and/or not during bowel movements.

5. The method according to claim 3 wherein the questions about the symptom or sign prompt responses relating to the symptom or sign at its best and at its worst.

6. The method according to claim 1 wherein the calculation produces mean and standard deviation of unweighted scores based on the responses from the patient.

7. The method according to claim 1 further comprising characterizing severity of the hemorrhoid disease into categories of at least severe, moderate, and mild.

8. The method according to claim 1 wherein the calculation produces a score along a continuum within an available range of the responses.

9. The method according to claim 2 performed at a beginning and end of a study period; and further comprising taking at least one physical measurement of afflicted bodily tissues as at least part of the objective measurement at the beginning and the end of the study period, wherein correlating the result of the calculation includes correlating the patient reported output information at the beginning and end of the study period with the at least one physical measurement of the afflicted bodily tissues.

10. The method according to claim 9 wherein a treatment of the hemorrhoid disease is applied during the study period, and further comprising determining if the afflicted bodily tissues correlate to progression of the hemorrhoid disease based on multiple physical measurements of the afflicted bodily tissues.

11. The method according to claim 10 wherein the at least one physical measurement includes a measurement of blood flow through the hemorrhoidal vein or artery.

12. The method according to claim 11 wherein the at least one measurement includes a Doppler measurement to quantify size, shape, and volume at the beginning and end of the study period.

13. The method according to claim 2 further comprising reporting the patient reported outcome information in a manner enabling selection of a treatment profile.

14. The method according to claim 1 performed on multiple test patients that have been administered a drug or treated with a device and performed on multiple patients of a control population that have not been administered the drug or treated with the device, and wherein the method further comprises reporting patient reported outcome information for both the multiple test patients and the multiple patients of the control population to a regulatory body responsible for approving the drug or device.

15. A method for assessing a state of hemorrhoidal disease, the method comprising:
a) observing varicosity in a region of tissues afflicted by the hemorrhoidal disease in a patient with a hemorrhoidal vein and artery;
b) measuring pressure and blood flow in the hemorrhoidal vein or artery over a length, width, and volume of the vein or artery; and
c) assessing a state of the hemorrhoidal disease as a function of at least one of the varicosity, pressure, and blood flow.

16. The method according to claim 15 performed at a beginning and an end of a treatment protocol period.

17. The method according to claim 16 further comprising:
a) obtaining, from the patient, responses to a patient reported output measure directed to the hemorrhoidal disease;
b) categorizing severity of the hemorrhoidal disease as a function of the patient responses to determine a category of the severity; and
c) comparing the state of the hemorrhoidal disease to the category of the severity.

18. The method according to claim 17 further comprising recommending a treatment plan as a function of the results of the comparing.

19. A computer-implemented method for assessing health of a patient suffering from hemorrhoid disease, the method comprising:
a) enabling a patient to provide at least partial responses to a patient reported outcome measure (PROM) about hemorrhoid disease via an electronic medium, the PROM including questions extending beyond a question about a symptom or sign of hemorrhoid disease;
b) performing a calculation at the electronic medium, or at a server coupled to the electronic medium, as a function of responses to the questions; and
c) correlating a result of the calculation with an objective measurement of the symptom or sign to produce patient reported outcome information for assessing the health of the patient.
